(19) United States
(12) Patent Application Publication
(10) Pub. No.: US 2007/0184502 A1
(43) Pub. Date: Aug. 9, 2007

(54) Method of diagnosing Sjogren's syndrome

(76) Inventors: Torsten Matthias, Flonheim (DE); Torsten Witte, Hannover (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/406,524

(22) Filed: Apr. 19, 2006

Related U.S. Application Data
(63) Continuation of application No. 11/198,896, filed on Aug. 8, 2005, now abandoned.

(30) Foreign Application Priority Data
Aug. 20, 1999 (DE) 199 39 575.5

(51) Int. Cl. G01N 33/53; C12M 1/34

(52) U.S. Cl. 435/7.92; 435/287.2

(57) ABSTRACT

The invention relates to a method of diagnosing Sjögren's syndrome and to a suitable kit of reagents.

The invention relates to a method of diagnosing Sjögren's syndrome and to a reagent kit suitable therefor.

Sjögren's syndrome is a chronic systemic disorder characterized by dryness of the eyes (kerato-conjunctivits, sicca), of the mouth (xerostonia sicca) and of other mucous membranes. If only the eyes and the mouth are affected by Sjögren's syndrome, the term used is primary Sjögren's syndrome. Secondary Sjögren's syndrome is complicated by the additional occurrence of various rheumatoid diseases, e.g. rheumatoid arthritis scleroderma and lupus erythematosus. The prevalence of the disorder varies between about 0.1 and 0.5% of the population.

Sjögren's syndrome is diagnosed according to certain classification criteria which encompass

(1) ocular symptoms,

(2) oral symptoms,

(3) ocular findings, i.e. positive Schirmer or Rose-Begal test,

(4) histological findings,

(5) findings on the salivary gland, for example based on salivary gland biopsy and

(6) detection of autoantibodies, e.g. anti-Ro/SSA or anti-La/SSB, antinuclear antibodies or rheumatoid factors.

In the USA it is necessary for all of the six criteria mentioned to be met for diagnosis of Sjögren's syndrome, whereas only four of these six criteria suffice in Europe.

Since autoantibodies play a large part in Sjögren's syndrome, attempts have been made for some years to find a specific marker for this disease. However, the autoantibodies mentioned above under item 6 are not specific and also occur in many other rheumatoid diseases.

Autoantibodies against α-fodrin have been detected in patients with Sjögren's syndrome (Haneji et al. Science 276 (1997), 604-607; Miyagawa et al., J. Invest. Dermatol. 111 (1998) 1189-1192 and Watanabe et al., Dermatol. 135 (1999), 535-539). α-fodrin autoantibodies of immunoglobulin class G have been found by immuno-blotting in patients with Sjögren's syndrome but also in patients with lupus erythematosus. These findings indicate that IgG antibodies against α-fodrin have no specific association with the presence of Sjögren's syndrome. In addition, the data were obtained on a relatively small number of patients. In one publication, a patient group of 43 patients with primary and 8 patients with secondary Sjögren's syndrome was tested. In all the subsequent publications, 9 patients with primary Sjögren's syndrome and 15 patients with secondary Sjögren's syndrome were tested.

In the inventors' own investigations on larger groups of patients (94 patients with Sjögren's syndrome, 352 patients with systemic lupus erythematosus and 160 control subjects) it was not possible to confirm the high specificity of IgG autoantibodies against α-fodrin for Sjögren's syndrome. It was possible to show instead that IgA autoantibodies against α-fodrin show a considerably higher specificity, namely 99.7% for primary Sjögren's syndrome.

The invention thus relates to a method of diagnosing Sjögren's syndrome, where the presence or/and the amount of IgA autoantibodies against α-fodrin is determined in a sample, which is normally derived from a patient to be investigated.

The occurrence of significant amounts of IgA autoantibodies against α-fodrin is highly specific for the presence of Sjögren's syndrome, especially primary Sjögren's syndrome, i.e. a false-positive diagnosis can be substantially precluded. The method moreover has a high sensitivity of about 70%, which may even be improved if—in addition to the IgA determination—the presence or/and the amount of autoantibodies of different immunoglobulin classes, e.g. IgG or/and IgM, against α-fodrin is determined. Thus, for example, the sensitivity of the method can be increased by 10% through an additional determination of IgG autoantibodies.

The method of the invention can be carried out as qualitative or quantitative determination. In a qualitative determination, IgA autoantibody concentrations which are above the so-called cutoff value are classified as positive. The cutoff value can be determined by calibrating the test system with positive and negative control samples. Alternatively, it is also possible to carry out a quantitative determination.

The sample to be tested is generally a human body fluid which is known possibly to contain IgA antibodies and, where appropriate, other antibodies. Examples of suitable body fluids are blood, serum, plasma or saliva, with serum being particularly preferred.

Determination of IgA antibodies against. α-fodrin can take place in accordance with any test formats known in the special field. Preference is given to the use of an α-fodrin antigen and of an IgA-specific receptor. The α-fodrin antigen may be a native protein which is obtainable from human cell lines, or a recombinant antigen which has been produced by recombinant protein expression in a heterologous host cell, e.g. a bacterial cell such as E. coli or a eukaryotic host cell such as an insect cell. Preference is given to the use of a recombinant α-fodrin antigen which comprises the sequence of native α-fodrin or parts thereof, in particular the N-terminal section. The recombinant antigen may additionally comprise heterologous peptide or polypeptide domains, e.g. a poly-His sequence which facilitates purification after expression.

The IgA-specific receptor is generally an antibody which is able selectively to recognize immunoglobulins of class A in the presence of immunoglobulins of other classes, e.g. G or/and M. It is possible to use for this purpose polyclonal anti-IgA antisera which are obtainable by immunization of experimental animals, e.g. goats, rats, mice, rabbits etc., with human IgA by known methods. However, it is likewise possible to employ corresponding monoclonal anti-IgA antibodies.

As already mentioned, the specific test format is not in general critical. However, preference is given to the use of a heterogeneous test format, particularly preferably a heterogeneous test format in which an immune complex consisting of α-fodrin antigen, IgA autoantibody to be detected and IgA-specific receptor is bound to a solid phase (sandwich test format). However, it is likewise possible to choose a competitive test format.

It is possible to use in a heterogeneous sandwich test format

(a) an α-fodrin antigen immobilized on the solid phase, and a labeled IgA-specific receptor or

(b) an IgA-specific receptor immobilized on the solid phase, and a labeled α-fodrin antigen.

Solid phases which can be employed are reaction vessels, microtiter plates, beads, biochips etc. The antigen or the receptor can be immobilized on the solid phase by adsorptive interactions, covalent bonding or mediated by a high-affinity binding pair (streptavidin/biotin, hapten/anti-hapten antibody). The immobilized test reagent can be employed in a form which is already bound to a solid phase or else be immobilized only during the test.

The method can be carried out as liquid test (e.g. in a reaction vessel) or else as dry test (e.g. on a test strip).

The labeled test reagent may itself have a detectable or signal-emitting group (direct labeling) or be capable of binding to a detectable group (indirect labeling). The labeling group can be selected as desired from all labeling groups known from the prior art for immunological detection methods, for example from enzymes, metal particles or latex particles, and luminescent or fluorescent groups. It is particularly preferred for the labeling group to be selected from enzymes, e.g. peroxidase, β-galactosidase or alkaline phosphatase, and for the method to be carried out in the ELISA format.

The invention further relates to a test kit for diagnosing Sjögren's syndrome, comprising

(a) an α-fodrin antigen and

(b) an IgA-specific receptor.

The test kit may additionally comprise (c) a solid phase onto which one of the test reagents (a) or (b) is bound or is capable of being bound. The test kit moreover preferably comprises (d) a labeling group which is bound to one of the test reagents (a) or (b) or is capable of being bound thereto.

The test kit may additionally comprise (e) at least one other antibody class-specific test reagent if the intention is, besides IgA autoantibodies, also to determine α-fodrin antibodies of other immunoglobulin classes. Examples of such antibody class-specific test reagents are anti-IgG antibodies or protein G for selective binding of IgG autoantibodies, or anti-IgM antibodies for selective binding of IgM autoantibodies. The test kit may additionally comprise other conventional reagents such as buffers, substrates and wetting solutions.

The invention is further to be made clear by the following examples:

### EXAMPLES

### 1. Material And Methods

1.1 Sera

Sera from patients with primary Sjögren's syndrome were collected in Austin, Tex. (n=49), Freiburg, Germany (n=20) and Hanover, Germany (n=18). Sjögren's syndrome was diagnosed in Austin in accordance with the US San Diego criteria and in Germany in accordance with the modified European criteria for the classification of Sjögren's syndrome.

Sera from patients with systemic lupus erythematosus (SLE) with or without secondary Sjögren's syndrome were obtained in Hanover. Secondary Sjögren's syndrome was diagnosed using the modified European classification criteria.

In addition, aliquots of 352 deep-frozen serum samples which had previously been collected for a German SLE investigation were used. All 352 patients met at least four of the ACR criteria for diagnosing SLE (Tan et al., Arthr. and Rheum. 25 (1982), 1271-1277). Overlapping syndromes were precluded. The patients had been characterized in detail in relation to their clinical and laboratory parameters in earlier investigations. Since some of these parameters had not been determined for all 352 patients, the correlations were carried out with the available data, using data from at least 339 patients for each clinical parameter and data from at least 1 patient for each laboratory parameter.

160 sera from blood donors were investigated as control of the specificity of the detection method.

1.2 Detection of Antibodies Against α-Fodrin By An ELISA

The cDNA for the N-terminal section of α-fodrin was cloned from the mRNA isolated from a human salivary gland by PCR. The primers had the position 93-130 (upstream) and 1827-1882 (downstream), resulting in a construct with 1731 bp (numbering corresponding to Moon et al., J. Biol. Chem. 265 (1991) 4427-4433). The cDNA was cloned into prokaryotic and eukaryotic expression vectors (respectively pet32b (Novagen) and pVL 1393 (Pharmingen)) and expressed in the form of a His tag fusion protein in E. coli and Sf9 insect cells. The recombinant protein was used to coat ELISA plates.

The sera were diluted 1:100 in a dilution buffer of pH 7.4 (75 mM NaCl, 0.1% Tween 20). 100 μl of the diluted sera were incubated on the ELISA plates for 30 min. After 3 washing steps with dilution buffer using an automated ELISA washer (SLT Labinstruments, Grödig, Austria), goat antiserum which was specific for IgG or IgA and was labeled with horseradish peroxidase was added for 15 min. After three further washing steps with dilution buffer, 100 μl of tetramethylbenzidine were added as substrate for a period of 15 min. The reaction was stopped by adding 100 μl of 1 M HCl, and the extinction (OD) at 450 nm was determined using an ELISA analyzer (Rainbow Reader SLT-Labinstruments, Grödig, Austria).

In order to determine a standard for the ELISA, ten sera from patients with primary Sjögren's syndrome were measured. The concentration of α-fodrin antibodies in the serum with the highest OD was arbitrarily defined as 100 U/ml. This serum was used as laboratory standard.

For daily calibration, this reference serum was used in concentrations corresponding to 0, 12, 5, 25, 50 and 100 U/ml. In order to obtain a standard curve, the measured OD values were plotted in a logarithmic/linear scale.

1.3 Detection of Rheumatoid Factors And of Autoantigens Ro And La

IgG, IgA and IgM rheumatoid factors, and autoantibodies against antigens Ro and La, were determined using commercially available ELISA systems in accordance with the manufacturer's methods (ORGen Tec GmbH, Mainz, Germany).

1.4 Statistical Analysis

The presence of IgG and IgA antibodies against α-fodrin in the 352 sera obtained from SLE patients was correlated with the clinical and laboratory parameters.

Non-parametric tests were used for the statistical analysis because the distribution of rheumatoid factors clearly deviated from a Gaussian distribution. Correlation of antibodies against α-fodrin with clinical and laboratory parameters was determined using the chi-square test. A probability of association of less than 0.05 was regarded as statistically significant.

### 2. Results

2.1 Distribution of IgA And IgG Antibodies Against α-Fodrin In Patients' And Control Samples

In 160 sera from blood donors, the mean (± standard deviation) of the concentration of IgA and IgG antibodies against α-fodrin was respectively 9.2 U/ml±5.5 U/ml and 11.1 U/ml±7.2 U/ml. The percentage of sera with antibodies against α-fodrin was calculated using a cutoff value which was defined as mean concentration of antibodies against α-fodrin in the sera from blood donors plus 3 standard deviations (corresponding to 25 U/ml for IgA antibodies and 32 U/ml for IgG antibodies).

Using the ELISA test described in 1.2, IgA antibodies against α-fodrin were identified in 53 of the 83 sera from patients with primary Sjögren's syndrome (64%). IgA antibodies against α-fodrin were likewise found in 7 of 15 sera from patients with SLE and Sjögren's syndrome (47%).

IgA antibodies against α-fodrin were detectable only in one of 160 blood donor sera and in one of 50 sera from SLE patients without Sjögren's syndrome. This revealed a test specificity of 99.7%.

IgG antibodies against α-fodrin were detected in 48 of 83 sera from patients with primary Sjögren's syndrome (57%). It was likewise possible to find IgG antibodies against α-fodrin in 6 of 15 sera from patients with SLE and secondary Sjögren's syndrome (40%).

IgG antibodies against α-fodrin were also detected in 3 of 160 blood donor sera and in none of 50 sera from SLE patients without Sjögren's syndrome.

10% of the patients with primary Sjögren's syndrome had only IgG antibodies, but no IgA antibodies, against α-fodrin.

It was not possible to find any correlation between the presence of IgG antibodies and IgG anti-α-fodrin antibodies with autoantibodies against Ro, La and IgA or IgG rheumatoid factors in the 83 patients with primary Sjögren's syndrome.

2.2 Association of IgA And IgG Antibodies Against α-Fodrin With SLE Clinical Parameters

The presence of IgA antibodies against α-fodrin showed a positive correlation with the parameters of erythema (p<0.01) and fetal loss (p<0.05). The presence of IgG antibodies against α-fodrin correlated with cutaneous vasculitis (p<0.05) and arthralgia (p<0.05).

2.3 Association of IgG And IgA Antibodies Against α-Fodrin With SLE Laboratory Parameters

The presence of IgA antibodies against α-fodrin showed a positive correlation with the parameters of increased IgA concentration (p<0.001), increased IgM concentration (p<0.05), neutropenia (p<0.05), IgG antibodies against cardiolipin (p<0.01), IgA (p<0.001) and IgM (p<0.05) autoantibodies against β2 glycoprotein and IgG rheumatoid factors (p<0.05). IgG antibodies against α-fodrin correlated only weakly with a positive Critidie reaction (p<0.05).

1. A method of diagnosing Sjögren's syndrome, characterized in that the presence or/and the amount of IgA autoantibodies against α-fodrin is determined in a sample derived from a patient.

2. The method as claimed in claim 1, characterized in that a human body fluid, in particular blood, serum, plasma or saliva, is used as sample.

3. The method as claimed in claim 1 or 2, characterized in that an α-fodrin antigen and an IgA-specific receptor are used for determining the IgA autoantibodies.

4. The method as claimed in any of claims 1 to 3, characterized in that a heterogeneous test format is used.

5. The method as claimed in claim 4, characterized in that a sandwich test format is used.

6. The method as claimed in any of claims 4 to 5, characterized in that
(a) an immobilized α-fodrin antigen and a labeled IgA-specific receptor or
(b) an immobilized IgA-specific receptor and a labeled α-fodrin antigen are used.

7. The method as claimed in claim 6, characterized in that the labeling group is selected from enzymes, metal particles or latex particles and luminescent or fluorescent groups.

8. The method as claimed in any of the preceding claims, characterized in that the presence or/and the amount of autoantibodies of other immunoglobulin classes against α-fodrin is additionally determined.

9. The method as claimed in claim 9, characterized in that IgG or/and IgM autoantibodies against α-fodrin are additionally determined.

10. A test kit for diagnosing Sjögren's syndrome, comprising
(a) an α-fodrin antigen and
(b) an IgA-specific receptor.

11. The test kit as claimed in claim 10, additionally comprising
(a) a solid phase onto which one of the test reagents (a) or (b) is bound or can be bound.

12. The test kit as claimed in claim 10 or 11, additionally comprising
(d) a labeling group which is bound to one of the test reagents (a) or (b) or can be bound thereto.

13. The test kit as claimed in any of claims 10 to 12, additionally comprising
(e) at least one other antibody class-specific test reagent.
